(19) United States
(12) Patent Application Publication
(10) Pub. No.: US 2025/0127775 A1
(43) Pub. Date: Apr. 24, 2025

(54) METHOD OF INDUCING DENDRITIC AND SYNAPTIC GENESIS IN NEURODEGENERATIVE CHRONIC DISEASES

(71) Applicant: Robert John Petcavich, Montgomery, TX (US)

(72) Inventor: Robert John Petcavich, Montgomery, TX (US)

(21) Appl. No.: 18/829,214

(22) Filed: Sep. 9, 2024

Related U.S. Application Data
(63) Continuation of application No. 17/254,534, filed on Dec. 21, 2020, now Pat. No. 12,083,116.
(60) Provisional application No. 62/687,825, filed on Jun. 21, 2018.

(51) Int. Cl. A61K 31/48; A61K 9/00; A61K 9/70; A61K 31/05; A61K 31/12; A61K 31/137; A61K 31/15; A61K 31/155; A61K 31/352; A61K 31/353; A61K 31/355; A61K 31/36; A61K 31/375; A61K 31/4045; A61K 31/426; A61K 31/675; A61K 31/706; A61K 45/06; A61P 25/28

(52) U.S. Cl. CPC A61K 31/48; A61K 9/006; A61K 9/7007; A61K 31/05; A61K 31/12; A61K 31/137; A61K 31/15; A61K 31/155; A61K 31/352; A61K 31/353; A61K 31/355; A61K 31/36; A61K 31/375; A61K 31/4045; A61K 31/426; A61K 31/675; A61K 31/706; A61K 45/06; A61P 25/28

(57) ABSTRACT

The present invention discloses a method to recover and restore dendritic and synaptic neuron connections that have been degraded or destroyed by neurodegenerative diseases. In the present invention tryptamines are used to induce neuro plasticity and restore both dendritic density and synaptic connections of neurons in the brain. In the preferred embodiment LSD given in micro doses can induce dendritic and synaptic genesis in neuronal networks and improve the quality of life of people with neurodegenerative diseases such as Alzheimer's, Huntington's, Multiple Sclerosis, Parkinson's and Frontotemporal dementia.

### CROSS-REFERENCE TO RELATED APPLICATIONS

This application is a continuation of U.S. application Ser. No. 17/254,534 filed on Dec. 21, 2021, which is a 35 U.S.C. § 371 continuation of International Application No. PCT/US2019/038486, filed on Jun. 21, 2019, that claims priority to U.S. Provisional Patent Application No. 62/687,825 filed on Jun. 21, 2018. The entire contents thereof are incorporated by reference herein.

### INCORPORATION BY REFERENCE

The content of the electronically submitted sequence listing, file name: PETC_002_US_CON.xml; size: 9.18 kilobytes; and date of creation: Sep. 9, 2024, filed herewith, is incorporated herein by reference in its entirety.

### BACKGROUND OF THE INVENTION

During the past 20 to 30 years a significant amount of research has been conducted on neurodegenerative diseases. As the population has aged worldwide, debilitating neurological disease such as Alzheimer's, Parkinson's, Frontotemporal Dementia and others have caused a significant emotional and financial burdens on developed and developing societies. As the world population ages, these afflictions will only increase and tax the current medical infrastructure to the breaking point. A solution is desperately needed to not only reduce the financial costs, but also enhance the quality of life for the millions of patients afflicted with these terrible chronic conditions.

Senile dementia has a tendency to increase with the shift to an aging society. This tendency has become a major large social problem. Dementia syndromes such as Frontal Temporal Dementia (FTD) and Alzheimer disease (AD) have their own pathologic characteristics including degeneration and loss of synaptic connections and neurons in certain brain areas, such as the cortical and cholinergic neurons.

Alzheimer's disease (AD) affects 5% of people over age 65 years, and its prevalence is increasing. Although AD manifests amyloid plaques and tau tangles, loss of synapses, eventually accompanied by neuronal loss, more closely correlates with cognitive decline. Damage to neurons occurs at least partially through generation of oxidative and nitrosative stress, due to excessive generation of reactive oxygen/nitrogen species (ROS/RNS) triggered by oligomeric amyloid-(Aβ) peptide.

Alzheimer's disease (AD) is a chronic neurodegenerative disease that usually starts slowly and worsens over time. It is the cause of 60-70% of cases of dementia. The most common early symptom is difficulty in remembering recent events (short-term memory loss). As the disease advances, symptoms can include problems with language, disorientation (including easily getting lost), mood swings, loss of motivation, not managing selfcare, and behavioral issues. As a person's condition declines, they often withdraw from family and society. Gradually, bodily functions are lost, ultimately leading to death. Although the speed of progression can vary, the typical life expectancy following diagnosis is three to nine years.

The cause of Alzheimer's disease (AD) is poorly understood. About 70% of the risk is believed to be genetic with many genes usually involved. Other risk factors include a history of head injuries, depression, or hypertension. The disease process is associated with plaques and tangles in the brain. A probable diagnosis is based on the history of the illness and cognitive testing with medical imaging and blood tests to rule out other possible causes. Initial symptoms are often mistaken for normal ageing. Examination of brain tissue is needed for a definite diagnosis. Mental and physical exercise, and avoiding obesity may decrease the risk of AD; however, evidence to support these recommendations is not strong. There are currently no medications or supplements that have been shown to decrease risk.

No treatments stop or reverse the progression of Alzheimer's disease (AD), although some may temporarily improve symptoms. Affected people increasingly rely on others for assistance, often placing a burden on the caregiver; the pressures can include social, psychological, physical, and economic elements. Exercise programs may be beneficial with respect to activities of daily living and can potentially improve outcomes. Behavioral problems or psychosis due to dementia are often treated with antipsychotics, but this is not usually recommended, as there is little benefit with an increased risk of early death.

During the 2002 to 2012 observation period, 413 Alzheimer's disease (AD) trials were performed: 124 Phase 1 trials, 206 Phase 2 trials, and 83 Phase 3 trials. Seventy-eight percent were sponsored by pharmaceutical companies. The United States of America (U.S.) remains the single world region with the greatest number of trials; cumulatively, more non-US. than U.S. trials are performed. The largest number of registered trials addressed symptomatic agents aimed at improving cognition (36.6%), followed by trials of disease-modifying small molecules (35.1%) and trials of disease-modifying immunotherapies (18%). The mean length of trials increases from Phase 2 to Phase 3, and the number of participants in trials increases between Phase 2 and Phase 3. Trials of disease-modifying agents are larger and longer than those for symptomatic agents. A very high attrition rate was found, with an overall success rate during the 2002 to 2012 period of 0.4% (99.6% failure).

In 2015, there were approximately 29.8 million people worldwide with Alzheimer's disease (AD). It most often begins in people over 65 years of age, although 4% to 5% of cases are early-onset Alzheimer's which begin before this. It affects about 6% of people 65 years and older. In 2015, dementia resulted in about 1.9 million deaths. It was first described by, and later named after, German psychiatrist and pathologist Alois Alzheimer in 1906. In developed countries, AD is one of the most financially costly diseases.

Therefore, it is obvious based on all the clinical trial failures of the past that a different strategy is needed to combat such insidious diseases such as Alzheimer's disease (AD). Since none of the prior therapeutic strategies has worked, it is time for a novel approach to treat chronic neurodegenerative diseases as disclosed in the present invention which is to restore both dendritic and synaptic connections in patients with non-addictive tryptamine molecules.

### BRIEF DESCRIPTION OF FIGURES

FIG. 1 shows an image of rat neurons, dendrites and synaptic connections that have been compromised by a neurodegenerative disease toxic A protein; and

FIG. 2 shows an image sequence of cortical rat neurons, with regenerated dendrites and synaptic connections after exposure to various tryptamine small molecules.

### SUMMARY OF THE INVENTION

The object of the present invention is to restore and regenerate neuron dendritic density and synaptic connections in humans with the application of tryptamine molecules.

Another object of the present invention is to restore and regenerate neuron dendritic and synaptic connections in humans with appropriate doses of the tryptamine lysergic acid (otherwise known as Lysergic acid diethylamide or LSD).

Another object of the present invention is to restore and regenerate neuron dendritic and synaptic connections in humans with appropriate doses of the tryptamines and phenethylamines selected from the group, N, N-dimethyltryptamine, 5-methoxy-N, N-dimethyltryptamine, mescaline, psilocin, 3,4-methylenedioxymethamphetamine, and psilocybin.

These and other objects of the invention will be elucidated in the detailed description of the invention.

### DEFINITIONS

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

Within the framework of the present description and in the subsequent claims, except where otherwise indicated, all numbers expressing amounts, quantities, percentages, and so forth, are to be understood as being preceded in all instances by the term “about”. As used herein, the term “about” is defined as ±5%. Also, all ranges of numerical entities include all the possible combinations of the maximum and minimum numerical values and all the possible intermediate ranges therein, in addition to those specifically indicated hereafter.

The term “and/or” as used herein is defined as the possibility of having one or the other or both. For example, “A and/or B” provides for the scenarios of having just A or just B or a combination of A and B. If the claim reads A and/or Band/or C, the composition may include A alone, B alone, C alone, A and B but not C, B and C but not A, A and C but not B or all three A, B and C as components.

The term “active form” or “active agent” herein refers to the metabolite form of the inactive prodrug that is metabolized within the body into its active form, regardless of the source of said prodrug.

The term “pharmaceutically acceptable salts or derivatives” herein refers to those salts or derivatives which possess the biological effectiveness and properties of the salified or derivatized compound and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate; hydrogen sulphate; citrate, maleate, fumarate, tifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCR, 127-133, 2008, herein incorporated by reference. The pharmaceutically acceptable derivatives include the esters, the ethers and the N-oxides.

### DETAILED DESCRIPTION OF THE INVENTION

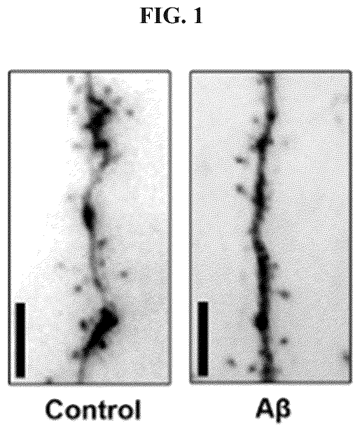

Referring to FIG. 1, this shows scanning electron microscope (SEM) images of rat neurons the black bar represents 5-micron scale. The control sample shows neurons of healthy rats before exposure to toxic A 42 amyloid proteins. At this resolution one can see copious dendritic structures, the round dark circles. The resolution is not high enough to visualize synaptic connections. The image labeled A is of rat neurons after exposure for 4days to 250 micro molar concentrations of toxic peptide. As can be seen in the image there is a significant degradation of the dendrites and although not seen the synaptic connections as well. This model emulates what is seen in the human versions of AD disease.

Autopsy results done by DeKosky et al. (Ann Neurol., 1990, 27 (5): 457-64) on patients that have died from Alzheimer's disease (AD) disease evidence a significant decrease in synaptic connections in their brains. The DeKosky results are summarized in Table I below. This is consistent with other publications showing that the number of neurons lost in AD patients at death is between 40 to 80%.

TABLE 1

| | Control | Biopsy AD | Autopsy AD |
|---|---|---|---|
| | Samples | Specimens | Specimens |
| Synapses/mm³ × 10⁸ | 3.81 ± 0.2 | 2.48 ± 0.16 | 2.22 ± 0.12 |
| | | −35%^{a} | −42%^{a} |
| Length of apposition | 0.350 ± 0.01 | 0.448 ± 0.01 | 0.431 ± 0.01 |
| (synaptic size) (μm) | | +28%^{a} | +23%^{a} |
| Total synaptic contact | 0.365 ± 0.02 | 0.386 ± 0.01 | 0.320 ± 0.01 |
| area per unit of | | +5% | −12.3%^{b,c} |
| volume (μm²/mm³) | | | |

There is a substantial body of evidence that inflammation causes neurodegenerative diseases, in particular Alzheimer's disease (AD). Human aging is a ubiquitous complex phenomenon that results from environmental, stochastic, genetic, and epigenetic events in different cell types and tissues and their interactions throughout life. A pervasive feature of aging tissues and most, if not all age-related diseases, is chronic inflammation. “Inflammaging” describes the low-grade, chronic, systemic inflammation in aging, in the absence of overt infection (“sterile” inflammation) and is a highly significant risk factor for both morbidity and mortality in elderly people. There is overwhelming epidemiological evidence that a state of mild inflammation, revealed by elevated levels of inflammatory biomarkers such as C-reactive protein and interleukin-6 (IL-6), is associated and predictive of many aging phenotypes—for example, changes in body composition, energy production and utilization, metabolic homeostasis, immune senescence, and neuronal health. The etiology of inflammation and its potential causal role in contributing to adverse health outcomes remains largely unknown. The identification of pathways that control age-related inflammation across multiple systems is therefore important in order to understand whether treatments that modulate Inflammaging may be beneficial in old people.

It is generally known that some of the causes of most neurodegenerative diseases, as a person ages, can be arrested with antioxidants and therapeutics. However, it may not be possible to treat all causes, therefore it is necessary to adopt a strategy to regenerate and restore neurons, dendrites and synapses.

It has been recently reported by Ly et al. (Cell Rep., 2018, 23(11):3179-82), that psychedelics promote structural and functional neural plasticity. It has been reported that certain psychedelics can promote changes in neuronal structure across vertebrates (rats) and invertebrates (Drosophila) species suggesting that they act through an evolutionary conserved mechanism. It is speculated that psychedelics may be used to treat depression, but there is no discussion or suggestion about efficacy of psychedelics to treat chronic neurodegenerative diseases.

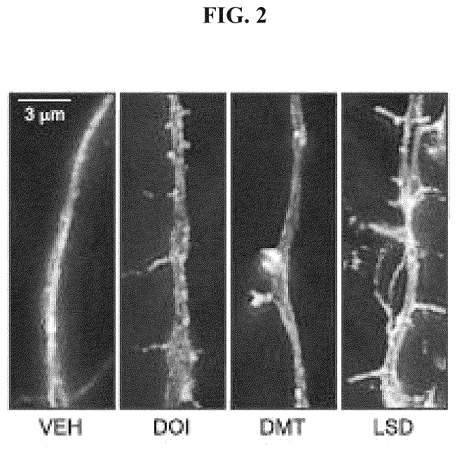

Referring to FIG. 2, the effect of various tryptamines on rat cortical dendrite and synaptic connections after 24 hours of exposure is demonstrated. As can be seen in the image, there is a dramatic increase in neurite and synapto genesis, particularly when the subject was treated with Lysergic acid diethylamide (LSD) having the formula:

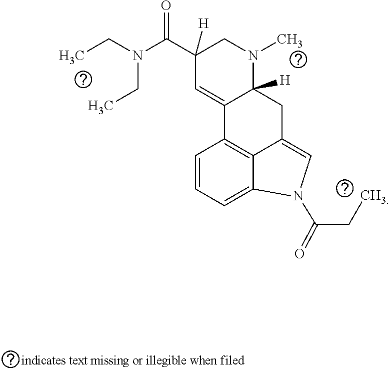 [SMILES: CCC(=O)n1cc2c3c(cccc31)C1=CC(C(=O)N(CC)CC)CN(C)[C@@H]1C2]

The effect is dramatic and fast even with very small doses of the compounds which is a desirable profile for a therapeutic molecule. Looking at FIG. 2 from left to right, it is apparent that LSD>DMT>DOI>Vehicle with regards to dendritic and synaptic generation. Potential therapeutics that can be used in the present invention are tryptamines such as tryptamines and phenethylamines selected from the group, N, N-dimethyltryptamine, 5-methoxy-N, N-dimethyltryptamine, Ketamine, noribogaine, psilocin 3,4-methylenedioxymethamphetamine, mescaline, psilocin, and psilocybin. The dose concentration will vary by molecule type but can range for 0.00001 to 250 milligrams to kilograms (mg/kg) of body weight with 0.01 to 50 mg/kg being preferred.

Tryptamines (also referred to herein as “tryptamine molecules), also known as serotonin analogues, are organic compounds which may be thought of as being derived from tryptamine,

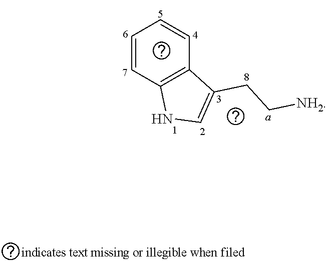 [SMILES: NCCc1c[nH]c2ccccc12]

The molecular structures of all tryptamines contain an indole ring, joined to an amino (NH2) group via an ethyl (-CH2-CH2-) sidechain. In substituted tryptamines, the indole ring, sidechain, and/or amino group are modified by substituting another group for one of the hydrogen (H) atoms. Well-known tryptamines include serotonin, an important neurotransmitter, and melatonin, a hormone involved in regulating the sleep-wake cycle. Tryptamine alkaloids are found in fungi, plants and animals; and sometimes used by humans for the neurological or psychotropic effects of the substance. Prominent examples of tryptamine alkaloids include psilocybin (from “Psilocybin mushrooms”) and DMT. Many synthetic tryptamines have also been made, including the migraine drug sumatriptan, and psychedelic drugs. The tryptamine structure, in particular its indole ring, may be part of the structure of some more complex compounds, for example: LSD, ibogaine, mitragynine and yohimbine. Preferred tryptamines, capable of practicing the claimed process, include, but are not limited to, N, N-dimethyltryptamine, 5-methoxy-N, N-dimethyltryptamine, mescaline, psilocin, 3,4-methylenedioxymethamphetamine, and psilocybin.

In order to better understand how to practice the invention an example of a therapeutic recommendation for patients is given below.

### EXAMPLE 1

LSD is always taken orally, and it is only injected for research purposes. It is commonly dissolved in blotting paper that can be broken down into tabs. Other, less common, ways of taking this psychedelic are consuming it in the form of a liquid or gelatin. The patient should place the tab under their tongue for around 10 minutes; after that, they can safely swallow the tab. The dose amount can vary by body weight and desired restorative effect, Low: 25-100 μg (people report feeling some effects with as little as 20 μg), Medium (common): 65-175 μg, High: 175-250 μg, and Heavy: 250+ μg for a typical body weight of 70 Kgs. In the present invention it is recommended to keep the dose equal to or less than medium to minimize any unwanted side effects or unpleasant psychedelic experiences.

The start time below is when the patient will usually begin to feel the effects of LSD from the time when the patient first ingests it. Duration is roughly the length of time the patient will experience the effects, after which the effects will start to wind down and they might start to feel the calm-down/comedown effects. Start 30-90 minutes, peak 2-3 hours and a duration of 9-14 hours. This depends heavily on the person and her/his state. Factors such as heart rate and blood pressure or the presence of other drugs in the blood affect the time the drug will take to have an effect. A higher heart rate and blood pressure than normal would make the effects to kick in faster. This can also vary with how much the patient has taken: the higher the dose taken by the patient, the longer the effect will last. Since this is intended for chronic use the lower the dose the better to maintain optimal neurite and synapto genesis.

Based on the example above it is expected that the average number of dendritic structures and synaptic branches will double for a typical neuron thereby restoring cognitive functions such as memory and spatial recognition that may have been compromised by disease progression.

As stated previously, the claimed method may also be practiced using phenethylamines either alone or in combination with tryptamines. Phenethylamine (PEA) is an organic compound, natural monoamine alkaloid, and trace amine, which acts as a central nervous system stimulant in humans. In the brain, phenethylamine regulates monoamine neurotransmission by binding to trace amine-associated receptor I (TAARI) and inhibiting vesicular monoamine transporter 2 (VMAT2) in monoamine neurons; to a lesser extent, it also acts as a neurotransmitter in the human central nervous system. The structure of phenethylamine is:

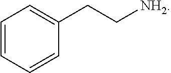

Phenethylamine is sold as a dietary supplement for purported mood and weight loss-related therapeutic benefits; however, in orally ingested phenethylamine, a significant amount is metabolized in the small intestine by monoamine oxidase B (MAO-B) and then aldehyde dehydrogenase (ALDH), which convert it to phenylacetic acid. This means that for significant concentrations to reach the brain, the dosage must be higher than for other methods of administration.

Substituted phenethylamines are a chemical class of organic compounds based upon the phenethylamine structure; the class is composed of all the derivative compounds of phenethylamine which can be formed by replacing, or substituting, one or more hydrogen atoms in the phenethylamine core structure with substituents. Many substituted phenethylamines are psychoactive drugs, which belong to a variety of different drug classes, including central nervous system stimulants (e.g., amphetamine), hallucinogens (e.g., 2,5-dimethoxy-4-methylamphetamine), entactogens (e.g., 3,4-methylenedioxyamphetamine), appetite suppressants (e.g. phentermine), nasal decongestants and bronchodilators (e.g., pseudoephedrine), antidepressants (e.g. bupropion), antiparkinson agents (e.g., selegiline), and vasopressors (e.g., ephedrine), among others. Many of these psychoactive compounds exert their pharmacological effects primarily by modulating monoamine neurotransmitter systems; however, there is no mechanism of action or biological target that is common to all members of this subclass. Numerous endogenous compounds—including hormones, monoamine neurotransmitters, and many trace amines (e.g., dopamine, norepinephrine, adrenaline, tyramine, and others)—are substituted phenethylamines. Several notable recreational drugs, such as 3,4-methylenedioxy methamphetamine (also known as MDMA or ecstasy), methamphetamine, and cathinones, are also members of the class. All of the substituted amphetamines are phenethylamines, as well. Pharmaceutical drugs that are substituted phenethylamines include phenelzine, phenformin, and fanetizole, among many others.

It may also be beneficial to supplement the method of dendritic and synaptic genesis with antioxidants that have a known effect on both brain and systemic inflammation such as Melatonin, Fisetin, Hydroxytyrosol, Carnosic Acid, Vitamin E, Vitamin C, Curcumin, Nicotinamide Mononucleotide, Tetrahydrocannabinol (THC) and Cannabidiol. The supplement dose can range from 25 to 2000 milligrams per day. An antioxidant supplement will help maintain and protect the new dendritic and synaptic formation from local inflammation.

The present invention also includes formulations used to treat neurodegenerative diseases utilizing the active agents as discussed above together with pharmaceutically acceptable carriers of said agents. The claimed formulations may also include one or more physiologically acceptable excipients. The term “physiologically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference. In particular, physiologically acceptable excipient, selected from the group comprising bulking agents, mixing agents, anticaking agents, aggregating agents or binders, lubricants, coating agents, stabilizers, natural flavorings, or a mixture thereof, preferably bulking agents, anticaking agents, stabilizers and natural flavorings and a mixture thereof, may be included in the claimed formulations.

Formulations as discussed herein may also contain a physiologically acceptable surfactant, buffer and/or preservative, provided said surfactant, buffer and/or preservative do not inhibit the therapeutic activity of any of the active agents. A “surfactant” as used herein is any compound that can greatly reduce the surface tension of water when used in very low concentrations. A “buffer” as used herein is any acid or salt combination which is pharmaceutically acceptable and capable of maintaining the composition of the present invention within a desired pH range. As used herein, a “preservative” is pharmaceutically acceptable, suitable for administration to a subject, which inhibits, prevents or delays the growth or microorganisms including, for example bacteria, viruses and fungi in the compositions of the present invention. Suitable preservatives for use in the compositions and methods of the present invention include, but are not limited to, cresols, benzyl alcohol, phenol, benzalkonium chloride, benzethonium chloride, chlorobutanol, phenylethyl alcohol, methyl paraben, propyl paraben, thiomersal and phenylmercuric nitrate and acetate, citric acid, sodium citrate, potassium sorbate, vitamin C (ascorbic acid), sodium ascorbate, sodium benzoate, potassium benzoate, grapefruit seed oil, or vegetable glycerin. In one embodiment, the preservative is m-cresol, chlorocresol or phenol.

### Kits

This disclosure also provides kits for conveniently and effectively implementing the methods disclosed herein. Such kits comprise any subject composition, and a means for facilitating compliance with methods disclosed herein. Such kits provide a convenient and effective means for assuring that the subject to be treated takes the appropriate active in the correct dosage in the correct manner. The compliance means of such kits includes any means which facilitates administering the actives according to a method disclosed herein. Such compliance means include instructions, packaging, and dispensing means, and combinations thereof. Kit components may be packaged for either manual or partially or wholly automated practice of the foregoing methods. In other embodiments involving kits, the disclosure contemplates a kit including compositions disclosed herein, and optionally instructions for their use.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The above discussion is meant to be illustrative of the principle and various embodiments of the present invention. Numerous variations, combinations and modifications will become apparent to those skilled in the art once the above disclosure is fully appreciated. It is intended that the following claims be interpreted to embrace all such variations and modifications.

I claim:

1. A method of inducing neuron dendritic and synaptic genesis in neurodegenerative diseases, comprising the steps of:
administering one or more tryptamine molecules, or pharmaceutically acceptable salts thereof to a patient suffering from a neurodegenerative disease, and
administering one or more phenethylamine molecules to said patient suffering from a neurodegenerative disease.

2. The method according to claim 1, wherein said neurodegenerative disease is a chronic condition selected from the group consisting of dementia, Alzheimer's disease, Parkinson's disease, frontal temporal dementia, Huntington's disease, psychosis due to dementia and multiple sclerosis; further wherein said Alzheimer's disease is the result of genetic predisposition, one or more head injuries, depression or hypertension.

3. The method according to claim 2, wherein said patient suffering from psychosis was previously treated with an antipsychotic drug and has stopped taking said antipsychotic drug prior to the administration of said one or more tryptamine molecules or pharmaceutically acceptable salts thereof.

4. The method according to claim 2, wherein said Alzheimer's disease is early onset Alzheimer's disease and/or the patient is 65 years of age or older and/or the neurodegenerative disease is caused by chronic inflammation.

5. The method according to claim 1, wherein said phenethylamine molecules are selected from the group consisting of amphetamines, hallucinogens, 2,5-dimethoxy-4-methylamphetamine, entactogens, 3,4-methylenedioxyamphetamine, phentermine, pseudoephedrine, bupropion, anti-Parkinson agents, selegiline, vasopressors, ephedrine, dopamine, norepinephrine, adrenaline, tyramine, 3,4-methylenedioxymethamphetamine, methamphetamine, cathinones, phenelzine, phenformin and fanetizole; and wherein said one or more tryptamine molecules is selected from the group consisting of lysergic acid diethylamide, N, N-dimethyltryptamine, 5-methoxy-N, N-dimethyltryptamine, mescaline, psilocin, 3,4-methylenedioxymethamphetamine, and psilocybin, pharmaceutically acceptable salts thereof and combinations thereof.

6. The method according to claim 1, further comprising the step of administering an antioxidant to said patient wherein said antioxidant administered to said patient is selected from the group consisting of melatonin, fisetin, hydroxytyrosol, camosic acid, vitamin E, vitamin C, curcumin, nicotinamide mononucleotide, tetrahydrocannabinol and cannabidiol; and wherein said one or more tryptamine molecules is selected from the group consisting of lysergic acid diethylamide, N, N-dimethyltryptamine, 5-methoxy-N, N-dimethyltryptamine, mescaline, psilocin, 3,4-methylenedioxymethamphetamine, and psilocybin, pharmaceutically acceptable salts thereof and combinations thereof.

7. The method according to claim 1, wherein said patient is administered said one or more tryptamine molecules or pharmaceutically acceptable salts thereof orally;
wherein said patient is administered a dose of said one or more tryptamine molecules or pharmaceutically acceptable salts thereof orally ranging from 0.01 mg/kg, from 25 μg to more than 250 μg, preferably from 65 μg to 175 μg; and wherein said dose administered to said patient is dependent on the body weight of the patient in need of treatment thereof.

8. The method according to claim 7, wherein said one or more tryptamine molecules or pharmaceutically acceptable salts thereof are contained on blotting paper divided into tabs; wherein said patient places one or more tab containing said one or more tryptamine molecules or pharmaceutically acceptable salts thereof under their tongue.

9. The method according to claim 7, wherein said one or more tryptamine molecules or pharmaceutically acceptable salts thereof are contained in liquid or gelatin.

10. The method according to claim 7, wherein said dose of said one or more tryptamine molecules, or pharmaceutically acceptable salts thereof, administered to the patient, is adjusted to minimize any unwanted side effects or unpleasant psychedelic experiences
